(19) United States
(12) Patent Application Publication
(10) Pub. No.: US 2008/0009050 A1
(43) Pub. Date: Jan. 10, 2008

(54) Regulation of acid metabolite production

(76) Inventors: Zdenek Pokluda, Bolatice (CZ); Josef Satke, Opava (CZ); Vladimir Vala, Ostrava (CZ); Josef Valik, Opava (CZ)

Correspondence Address: KENYON & KENYON LLP, ONE BROADWAY, NEW YORK, NY, 10004, US

(21) Appl. No.: 11/823,905

(22) Filed: Jun. 29, 2007

Related U.S. Application Data
(60) Provisional application No. 60/818,145, filed on Jun. 29, 2006.
(60) Provisional application No. 60/847,344, filed on Sep. 25, 2006.

(51) Int. Cl. C12P 1/02

(52) U.S. Cl. 435/171

(57) ABSTRACT

The present invention provides a method of preparing acid metabolites, such as mycophenolic acid, and a strain of Penicillium sp. with accession number CCM 8364 for use in such method, which method produces mycophenolic acid at increased yields.

### CROSS REFERENCE TO RELATED APPLICATIONS

The present application claims the benefit of the following U.S. Provisional Patent Application No. 60/818,145, filed Jun. 29, 2006 and 60/847,344, filed Sep. 25, 2006. The contents of these applications are incorporated herein by reference.

### FIELD OF THE INVENTION

The present invention relates to a method of regulating acid metabolite production by a cell of a microorganism by regulation of the level of oxygen saturation in a fermentation broth. In particular, the invention relates to a method of regulating mycophenolic acid production by Penicillium by regulation of the level of oxygen saturation in the culture medium, and to a newly isolated strain of Penicillium having accession number CCM 8364.

### BACKGROUND OF THE INVENTION

Mycophenolic acid (referred to as MPA) is a metabolite produced by several species of Penicillium (Imperial Chemical Industries GB 1 158 387 (1967), Lilly & Co Elli GB 1 593 208 (1978)) or by mutant strains obtained from Penicillium (Ajinimoto KK U.S. Pat. No. 4,452,891 (1981)). MPA has biological and pharmacological activities e.g. as an antimicrobial (Florey et al., 1946), an antitumor agent (Williams et al., J. Antibiot., 21, 463 (1968), and as an immunosuppressive agent (Mitsui, Suzuki, 1969). In therapy, mycophenolate mofetil, a 2-morpholinoethyl ester derivative of MPA is commonly used.

The chemical structure of MPA is:

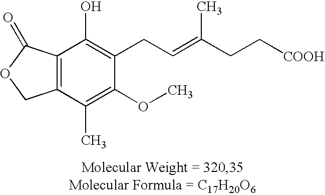

One of the methods to produce MPA is in a submerged culture fermentation, as described in EP 1 624 070 A1; wherein NaOH is used for regulating the pH of the fermentation medium, by keeping it between 4 and 7.

MPA may be isolated from the fermentation medium by a variety of processes including those disclosed in Imperial Chemical Industries GB 1158 387 (1967); BIOCON, WO 01/64931 A1 (2001); and IVAX, WO 2006/031665 (2006), WO 01/21607 and WO 2005/105768). The isolated MPA is then used for the production of mycophenolate mofetil (WO 02/100855).

A high level of MPA production is desirable for the efficient and economic production of drugs based on the active pharmaceutical ingredient mycophenolate mofetil. The present invention provides such a method.

### SUMMARY OF THE INVENTION

In one aspect, the present invention provides a method of regulating the production of an acid metabolite produced by a microbial cell, comprising regulating the oxygen saturation of a culture medium comprising the microbial cell. In a preferred embodiment, the method comprises providing acid metabolite producing microbial cells in a submerged fermentation culture system and producing the acid metabolite from the microbial cells in such culture, wherein the oxygen saturation of the culture medium is regulated to control pH of the culture medium. Preferably, the acid metabolite is mycophenolic acid (MPA).

In another aspect, the present invention provides a method of preparing mycophenolate mofetil comprising converting mycophenolic acid to mycophenolate mofetil, wherein the mycophenolic acid is prepared by regulating the oxygen saturation of a fermentation broth comprising a mycophenolic acid producing microbial cell.

In yet another aspect, the present invention further provides a strain of Penicillium sp. deposited as accession number CCM 8364 at Czech Collection of Microorganisms (CCM) at Masaryk University, Brno, Czech Republic. The strain of Penicillium sp. deposited as accession number CCM 8364 produces mycophenolic acid in high concentrations (in excess of 1 g MPA per liter of culture medium).

### BRIEF DESCRIPTION OF THE DRAWINGS

FIG. 1: A graphical representation displaying the regulation of culture medium pH, by regulating the level of oxygen saturation, to obtain a pH above pH 4.5.

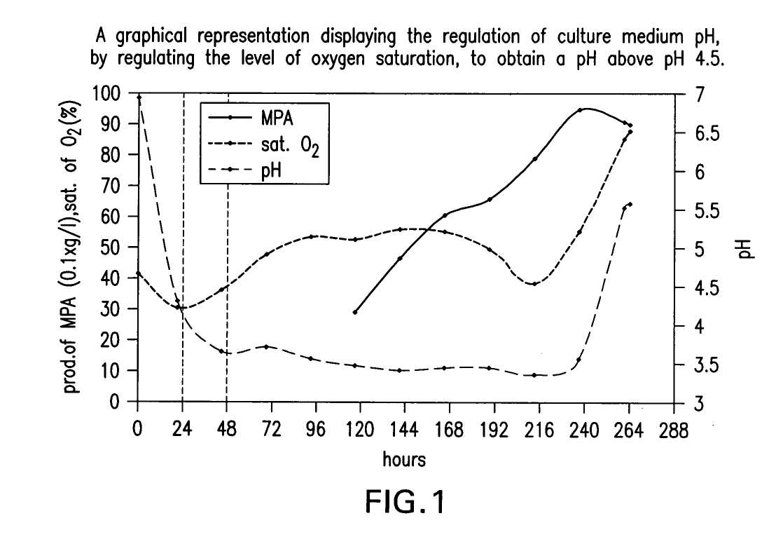

FIG. 2: A graphical representation displaying the regulation of culture medium pH, by regulating the level of oxygen saturation, to obtain a pH below pH 4.5.

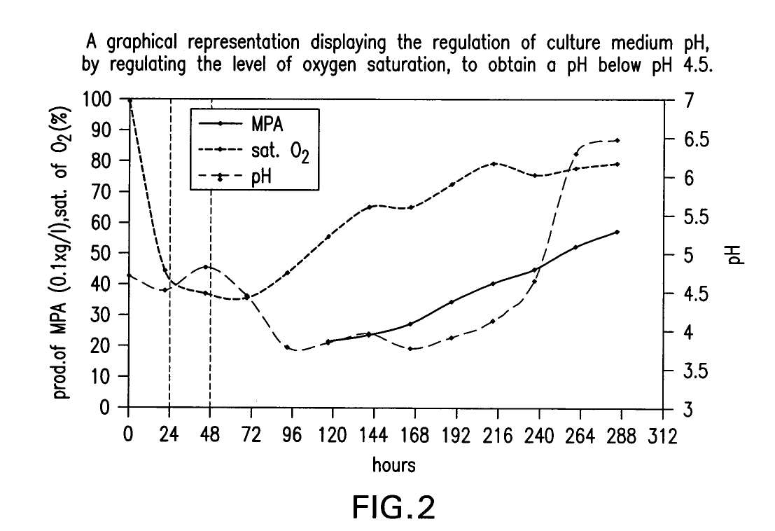

### DETAILED DESCRIPTION OF THE INVENTION

As used herein, the term “high levels” or “high concentrations” when related to MPA refers to a level (or concentration) of more than about 1 g of MPA per liter, preferably about 6 to about 10 g of MPA per 1 liter of fermentation broth (culture medium).

Several factors are important in obtaining high concentrations of MPA in the production medium of submerged culture fermentation. These factors include the composition of the medium and the cultivation conditions. In particular, an optimal pH of the fermentation broth has to be maintained during the production phase. Normally the addition of NaOH is used to regulate the pH of the fermentation broth and keep it between pH 4 and 7. NaOH is also used to produce a pH shift after the log phase of growth has ended. The present invention relates to a new method for regulating the production of MPA by regulating the pH of the fermentation broth/culture medium by regulating the level of oxygen without adding a chemical to the fermentation broth/culture medium. As such, the present invention uses only a natural source to regulate the pH of the fermentation broth/culture medium. Saturation by oxygen using air is performed by controlled aeration of this aerobic process, thereby regulating the pH of the process. Accordingly, the present process decreases consumption of hazardous acids and/or alkali compounds.

The present invention provides a method of regulating the production of an acid metabolite produced by a microbial cell, by regulating the oxygen saturation of the fermentation broth. More specifically, the method comprises providing a microbial cell in a submerged fermentation culture system, wherein the oxygen saturation of the culture medium of the submerged fermentation culture is regulated/controlled, to produce an acid metabolite. Regulating or controlling the oxygen saturation of such culture medium ensures that the pH of the culture medium is maintained between pH 4 and pH 7, preferably between pH 4 and pH 6, more preferably between pH 4 and pH 5.5. Such regulation provides a method of producing the acid metabolite obtaining a high concentration of the acid metabolite in the fermentation broth (culture medium).

Preferably, the acid metabolite is selected from the group consisting of uronic acid, paspalic acid, methylcarbinolamine of lysergic acid, and mycophenolic acid. Preferably the acid metabolite is mycophenolic acid.

Preferably, the microbial cell is selected from the group consisting of a fungal cell and a bacterial cell. Preferably the microbial cell is a fungal cell, more preferably the microbial cell is a Penicillium sp. cell for producing mycophenolic acid, a Claviceps sp. cell for producing paspalic acid and methylcarbinolamine, or a Klebsiella pneumoniae cell for producing uronic acid. Preferably, the Penicillium sp. cell is a single strain of Penicillium. Preferably, the strain of Penicillium is a strain having the accession number of CCM 8364.

In the method of the present invention a suitable culture medium comprises a basic and a complex nitrogen source. The basic nitrogen source may be for example simple inorganic molecules, amino acids or other organic compounds containing nitrogen, such as for example ammonia/ammonium salts, any naturally occurring amino acid. A preferred complex nitrogen source is hydrolyzed casein and/or corn steep. The culture medium also contains a carbon source. A suitable carbon source for use in the culture medium may be a carbohydrate, preferably a sugar, more preferably a monosaccharide, most preferably sucrose.

In addition, the culture medium comprises a specific amino acid supplement in addition to the complex nitrogen source, or where the basic nitrogen source is a simple amino acid. Preferably, the amino acid supplement includes one or more of glycine, methionine and asparagine. Preferable methionine is D,L-methionine. Preferably asparagine is L-asparagine.

The amino acid in the amino acid supplement is commonly used at a concentration of about 1 to about 30 g/l, preferably at a concentration of about 5 to about 8 g/l. Glycine is usually used at a concentration of about 1 to about 30 g/l, preferably glycine is used at a concentration of about 2 to about 20 g/l, more preferably glycine is used at a concentration of about 3 to about 15 g/l, even more preferably the concentration of glycine is about 5 to about 8 g/l.

D,L-methionine is commonly used at a concentration of about 0.01 to about 10 g/l, more preferably D,L-methionine is used at a concentration of about 0.1 to about 1 g/l.

L-asparagine is commonly used at a concentration of about 1 to about 30 g/l, more preferably L-asparagine is used at a concentration of about 2 to about 20 g/l, yet more preferably L-asparagine is used at a concentration of about 3 to about 15 g/l, even more preferably the concentration of L-asparagine is about 5 to about 8 g/l.

The regulation of MPA production occurs when the penicillium sp. is grown in a submerged fermentation culture having a defined oxygen saturation level. In such submerged fermentation culture the level of oxygen saturation in the culture medium is controlled during at least a part of the production phase. Alternatively the level of oxygen saturation is controlled throughout the entire production phase. As such the level of oxygen saturation in the culture medium may be controlled during the time period from 0 to 24 hours, or the time period from 0 to 48 hours, or from 24 to 48 hours or from 48 hours. Preferably, the level of oxygen saturation is controlled until the pH rises due to carbon source depletion at the end of the production phase.

The production of MPA in submerged fermentation may be increased by controlling the level of oxygen saturation in the culture medium. Preferably the level of oxygen saturation is in the range 5 to 40%, more preferably the level of oxygen saturation is in the range 5 to 35%, even more preferably the level of oxygen saturation is in the range from 5 to 30%. The % oxygen saturation refers to the pO₂ as measured by pO₂ electrode whereby 100% oxygen saturation is correlated to the maximal aeration of the culture medium without the microbial cell culture.

In a preferred embodiment, the Penicillium sp. is grown in a different culture medium in the inoculation and production stages of culture. The first stage (inoculation stage) starts at T=0 hours, a second stage may start at T=24 hours and the third stage (production stage) may start at T=48 hours.

Preferably, the process further comprises growing the microbial cells in an inoculating medium, and inoculating the production medium with an inoculum of at least 20%, preferably at least 25% of the volume of the production medium.

In the method of the present invention, the pH of the culture medium is maintained at a pH greater than pH 4, preferably between pH 4 and pH 7. Preferably, it is maintained by regulating the level of oxygen saturation in the culture medium. The level of oxygen saturation in the culture medium is regulated by continuous monitoring of the oxygen level using a pO₂ electrode and adjusting the oxygen level when necessary by adjusting the speed of agitation and added air volume. As such, the pH of the culture medium may be maintained at a pH greater than pH 4 during the production stage of the culture, which is the period at which time the acid metabolite is actively produced in the fermentation culture system. This period may start after an inoculate with the microbial cell has been transferred to the fermentation culture system. Preferably the pH is greater than pH 4.5 during the production stage of culture, more preferably the pH is between about pH 4.5 and about pH 5.5 during the production stage of culture.

The temperature of the submerged culture so regulated is maintained at about 20° C. to about 30° C. for a sufficient period of time to produce MPA. Preferably, the temperature of the submerged culture in the process of the present invention is maintained at about 24° C. to about 26° C. for a period of about 240 hours to about 300 hours.

The regulation of production of MPA according to the process of the present invention of regulating the level of oxygen saturation in the culture medium produces a final concentration of MPA (g/l of culture medium) that is in excess of 1 g/l, more preferably in excess of 2 g/l or in excess of 3 g/l, yet more preferably in excess of 4 g/l, even more preferably in excess of 5 g/l, in excess of 6g/1 or in excess of 8 g/l.

The present invention also provides a new strain of Penicillium sp. (deposited as accession number CCM 8364) that produces high levels of MPA, such as for example in excess of 10 g/l. This strain produces higher levels of MPA when compared to a wild type Penicillium sp. strain from which strain CCM 8364 was produced. Such wild type Penicillium sp. strain produces MPA with an average yield of about 600 mg/l when grown in submerged culture.

Also provided is a culture of such strain, preferably a biologically pure culture, such as one comprising only growing cells of the fungal strain with deposit No CCM8364. Such culture has preferably at least about 90% of such fungal strain. This strain in culture may be in the form of individual cells, cell aggregates, hyphas, filaments, and its aggregates in or on any surface or environment.

The strain of Pencillium sp. having accession number CCM8364 is capable to produce a final concentration of MPA (g/l of culture medium) that is in excess of 1 g/l, more preferably in excess of 2 g/l or in excess of 3 g/l, yet more preferably in excess of 4 g/l, even more preferably in excess of 5 g/l, in excess of 6 g/l or in excess of 8 g/l.

The Pencillium sp. strain, CCM 8364, is produced by using a system of chemical and physical mutation methods combined with positive selection media.

The present invention further provides a process for preparing mycophenolate mofetil by preparing mycophenolic acid by the process of the present invention, and further converting it to mycophenolate mofetil. Mycophenolic acid may be converted to mycophenolate mofetil, for example, according to the process disclosed in WO 02/100855.

Having thus described the invention with reference to particular preferred embodiments and illustrative examples, those skilled in the art can appreciate modifications to the invention as described and illustrated that do not depart from the spirit and scope of the invention as disclosed in the specification. The disclosures of the references referred to in this patent application are incorporated herein by reference. Further, the following examples are intended to further illustrate certain preferred embodiments of the invention and are not limiting in nature.

### EXAMPLES

### Example 1

### Growth Characteristics of Pencillium strain CCM 8364 Macroscopic Morphology

During saprophytic growth in agar-thickened malt medium—MEA—(Malt Extract Agar of 35 g malt extract, 5 g peptone, 13 g agar, 1000 ml H₂O), strain CCM 8364 forms colonies of sphacelus airy mycelium having a diameter of 1.1 cm after 7 days cultivation. The surface texture is velutinous, is centrally umbonate and has white margins. The level of conidiogenesis is moderate, the colonies are coloured near brown and have exudate present, buff, reverse buff.

During saprophytic growth in agar-thickened Czapek yeast medium, strain CCM 8364 forms colonies of sphacelus airy mycelium having a diameter of 2.3 cm after 7 days cultivation. The surface texture is velutinous, is radially sulcate and centrally umbonate with white margins. The level of conidiogenesis is moderate, the colonies are coloured near brown and have exudate present, red brown, reverse cinnamon.

During saprophytic growth in agar-thickened production medium G25N, strain CCM 8364 forms colonies of sphacelus airy mycelium having a diameter of 0.3 cm after 7 days cultivation. The surface texture is velutinous. The level of conidiogenesis is light to moderate, the colonies are centrally plane or are raised with white margins, coloured smoked grey—gloucous gray with greenish tinge, exudate absent, reverse pale.

Microscopic Morphology

The mycelium consist of stipes 300-600×4-6 μm, the surface is smooth, penicilli variable, terveticillate and irregular, with more branch points occurring in a non-uniform pattern, rami in verticils of 2-3, metulae in verticils, 12-20×4-4.5 μm, phialides ampulliform—acerose, measuring 13-16×2.5-3 μm, conidia in short chains, spheroidal 3.5-5.5 μm and ellipsoidal 4-6×2-4 μm with disjunctors, walls distinctly roughened.

### Example 2

### MPA Assay

Each isolate was evaluated for its MPA production. This assay consists of two steps—inoculation and production. The inoculation medium (IM) contains carbon sources (e.g. sucrose), nitrogen sources (suitable sources include hydrolyzed casein and corn steep) and inorganic salts (such as KH₂PO₄). 50 ml of IM was sterilized at 120° C. for 20 minutes in a 250 ml Erlenmeyer flask. The spores of the isolate were washed off an agar slant culture with 20 ml of sterile water. 5 ml of this spore suspension was used as the inoculum for IM and then cultivated for 3-5 days at 24-28° C. with a rate of stirring of 280 of revolutions/minute while shaking. The resulting IM was used for inoculation of the producing medium (PM) by the addition of 10% of the total volume. The PM, utilized in the present invention for the assay of MPA, contains carbon sources (e.g. sucrose), nitrogen sources (suitable sources include casein and amino acids as glycine, asparagine, methionine) and inorganic salts (e.g. K₃PO₄, MgSO₄). 50 ml of the PM was sterilized at 120° C. for 20 minutes in a 250 ml Erlenmeyer flask. A test cultivation was then carried out under aerobic conditions at 24-28° C. with shaking for 10 to 14 days. The rate of stirring was 280 revolutions/minute. The pH of the medium was measured before inoculation (pH 4.9 to pH 5.1) and two or three times before the end of the experiment using a pH meter as described above. The level of oxygen saturation was measured continuously in laboratory or plant fermenters. The MPA containing aliquots were assayed using HPLC chromatography according to standard techniques known to the person skilled in the art to determine their MPA content against a standard (internal IVAX standard MPA according Research report RR/RD/CH024/01-A, retest RR/RD/AN061/05-A). The column used was a Chromolith speed ROD, RP18e, 50×4.6 mm, (manufactured by MERCK), mobile phase: 40% acetonitrile, 60% water containing 680 1μl H₃PO₄/liter H₂0, flow 5-6.5 ml/min and temperature 30° C.

### Example 3

### Mutation and Selection Process

1. Selection

In the first stage of the procedure the sporulated slant of wild type Penicillium sp. was suspended in water and devoided of mycelia fragments. The suspension was then diluted to 100 spores/ml and spread on Petri plates containing MEA. The plates were then incubated at 24-27° C. for seven days. The colonies which grew on the agar plate were picked-off and sustained on slants of the same nutrient agar medium in a Blacke flask. After 16 days of cultivation monocolony isolates were tested in the production test. The isolate with the best production (1.2 g MPA/l in fermentation broth) was mutated in the next stage.

2. Mutating with N-methyl-N′-nitro-N-nitroso-guanidine (NG)

A sporulated slant was suspended in water and devoided of mycelia fragments. The conidia populations were then exposed to solution of 0.2M NG in saline for 8 hours. In this mutation treatment, approximately 99.9% of the spores were killed. The treated spores were then washed free of mutagen and spread on Petri plates containing MEA. The plates were incubated at 24-27° C. for seven days. The colonies that grew on the agar plate were picked-off and sustained on slants of the same nutrient agar medium in a Blacke flask. After 16 days of cultivation monocolony isolates were tested in the production test. The isolate with the best production (2.4 g MPA/l in fermentation broth) was mutated in the next stage.

3. Mutating with ethylmethanesulfonate (EMS)

A sporulated slant was suspended in water and devoided of mycelia fragments. The conidia populations were exposed to solution of 0.2M EMS in saline for 16 hours. In this mutation treatment approximately 99.9% of the spores were killed. The spores were then washed free of mutagen and inoculated on Petri plates containing MEA. The plates were incubated at 24-27° C. for seven days until colonies formed on the plates. The colonies were picked-off and sustained on slants of the same nutrient agar medium in a Blacke flask. After 16 days cultivation monocolony isolates were tested in the production test. The isolate with the best production (5.6g MPA/l in fermentation broth) was mutated in the next stage.

4. Mutating with UV Irradiation

A sporulated slant was suspended in water and devoided of mycelia fragments. The suspension was diluted to 1×10⁵ spores/ml and spread on Petri plates containing MEA. The plates were exposed to UV irradiation with the radiation dose being about 15 Jm⁻² s⁻¹ for about 500 seconds. In this mutation treatment approximately 99.9% of the spores were killed. The Petri plates were then incubated at 24-27° C. for seven days. The colonies that grew on the agar plate were picked-off and sustained on slants of the same nutrient agar medium in a Blacke flask. After 16 days of cultivation monocolony isolates were tested in the production test. The isolate with the best production (6.0 g MPA/l in fermentation broth) was mutated in the next stage.

5. Mutating with Gamma Irradiation

A sporulated slant was suspended in water and devoided of mycelia fragments. The suspension was diluted to 1×10⁵ spores/ml and exposed to Gamma radiation with radiation dose being 1500 Gy. Irradiated isolates were spread on Petri plates containing MEA. The Petri plates were then incubated at 24-27° C. for seven days until colonies formed on the plates The colonies were picked-off and sustained on slants of the same nutrient agar medium in a Blacke flask. After 16 days of cultivation monocolony isolates were tested in the production test. The isolate with the best production (10 g MPA/l in fermentation broth) was twice passaged on agar medium in a Blacke flask. After each passage the production test was performed to verify the continued high production of MPA.

This newly produced strain was deposited under the Budapest Treaty in the Czech Collection of Microorganisms (CCM) at the Masaryk University, Tvrdého 14, Bmo, Czech Republic under collection number CCM 8364.

The system of mutation procedures when combined with the positive selection procedures resulted in an increase in the average content of MPA from 600 mg MPA/l of fermentation broth for the parent strain to 10 g MPA/l of fermentation broth for strain CCM 8364 according to the MPA assay.

### Example 4

### MPA Production during Fermentation in High Oxygen Saturation Culture

Strain CCM 8364 was used for fermentation in a submerged fermenter with total volume about 14 cubic metres. The production medium contained sucrose as a carbon source and hydrolysed casein, glycine, asparagine and methionine as nitrogen sources and minerals including K, P and Mg at an initial pH of 5.0. The production medium was inoculated by a third stage inoculum (approximately 26% w/w). The cultivation temperature was 24-26° C. and the production fermentation run was 285 hours. The pH of cultivation medium was controlled by saturating it with oxygen to a level greater than 30%. The values of dissolved oxygen, pH during cultivation and the concentration of MPA at the end of production step are summarized in the following table. Due to oxygen saturation of the production media the pH dropped below pH 4 and the final MPA production level reached 5.7 g MPA/l.

| | | Time (h) | | | | | | | | MPA |
|---|---|---|---|---|---|---|---|---|---|---|
| No. | | 24 | 48 | 72 | 120 | 144 | 164 | 192 | 240 | g/l |
| 1 | Oxygen | 45% | 37% | 36% | 60% | 64% | 64% | 76% | 79% | 5.7 |
| | saturation | | | | | | | | | |
| | pH | 4.52 | 4.82 | 4.45 | 3.84 | 3.96 | 3.77 | 3.91 | 4.65 | |

### Example 5

### MPA Production during Fermentation in Medium Oxygen Saturation Culture

Strain CCM 8364 was used for fermentation in a submerged fermenter with total volume about 14 cubic metres. The production medium contained sucrose as a carbon source and hydrolysed casein, glycine, asparagine and methionine as nitrogen sources and minerals including K, P and Mg at an initial pH of 5.0. The production strain Penicillium sp. (CCM 8364) was used for fermentation. The production medium was inoculated by the third stage inoculum (approximately 26%, w/w). The cultivation temperature was 24-26° C. and the production fermentation run was 278 hours. The pH of cultivation medium was controlled by means of saturation with oxygen. The values of dissolved oxygen, the pH of the culture medium during cultivation and the concentration of MPA at the end of production step are given in the following table.

| | | Time (h) | | | | | | | | MPA |
|---|---|---|---|---|---|---|---|---|---|---|
| No. | | 24 | 48 | 72 | 120 | 144 | 164 | 192 | 240 | g/l |
| 2 | Oxygen | 24% | 18% | 17% | 24% | 20% | 21% | 18% | 16% | 7.5 |
| | saturation | | | | | | | | | |
| | pH | 4.24 | 4.54 | 4.77 | 5.15 | 5.37 | 5.14 | 4.47 | 4.41 | |

### Example 6

### MPA Production during Fermentation in Low Oxygen Saturation Culture

Strain CCM 8364 was used for fermentation in a submerged fermenter with total volume about 14 cubic metres. The production medium contained sucrose as a carbon source and hydrolysed casein, glycine, asparagine and methionine as nitrogen sources and minerals including K, P and Mg at an initial pH of 5.0. The production medium was inoculated by the third stage inoculum (approximately 26% w/w). The cultivation temperature was 24-26° C. The fermentation broth was fed by medium with a carbon source (1×10% w/w) at 143 hours and the production fermentation was finished at 257 hours. The pH of cultivation medium was controlled by means of saturation with oxygen. Values for dissolved oxygen, pH of the culture medium during cultivation and concentration of MPA in the end of production step are given in the following table.

| | | Time (h) | | | | | | | | MPA |
|---|---|---|---|---|---|---|---|---|---|---|
| No. | | 24 | 48 | 72 | 120 | 144 | 164 | 192 | 240 | g/l |
| 3 | Oxygen | 30% | 25% | 17% | 18% | 13% | 11% | 9% | 9% | 8.2 |
| | Saturation | | | | | | | | | |
| | pH | 4.03 | 4.19 | 4.65 | 5.05 | 5.09 | 5.06 | 4.86 | 5.65 | |

### Example 7

### Mycophenolate mofetil Production from Mycophenolic Acid, according to WO 02/100855.

100 g of crude mycophenolic acid, prepared according the example 5 were put in a reaction flask with a reflux cooler together with 200 ml dibutyl ether. Under continual stirring the mixture was warmed up to temperature of 50 to 60° C. and then 40 ml 2-morpholinoethanol were added. The reaction mixture was warmed up to boiling under azeotropic separation of water. After 48 hours the mixture was cooled up to 23° C. and 200 ml dichloromethane was added. The solution was extracted twice with 100 ml 0.5 M aqueous K₂CO₃ and once with 100 ml of water. Then dichloromethane was distilled off under vacuum and the suspension was cooled up to 10 to 15° C. In this matter synthetized crystalline mycophenolate mofetil was recrystallized from ethyl acetate. After drying the crystals 110 g mycophenolate mofetil was obtained with purity more than 99.0% (HPLC).

What is claimed is:

1. A method of regulating the production of an acid metabolite produced by a microbial cell, comprising regulating the oxygen saturation of a culture medium comprising the microbial cell.

2. The method according to claim 1, comprising providing acid metabolite producing microbial cells in a submerged fermentation culture system and producing the acid metabolite from the microbial cells in such culture, wherein the oxygen saturation of the culture medium is regulated to control pH of the culture medium.

3. The method according to claim 1, wherein the acid metabolite is selected from the group consisting of uronic acid, paspalic acid, methylcarbinolamine of lysergic acid, and mycophenolic acid.

4. The method according to claim 3, wherein the acid metabolite is mycophenolic acid (MPA).

5. The method according to claim 4, wherein the mycophenolic acid is produced in the culture medium to a final concentration of MPA (g/l of culture medium) that is in excess of 1 g/l.

6. The method according to claim 5, wherein the concentration is in excess of 5 g/l.

7. The method according to claim 6, wherein the concentration is in excess of 8 g/l.

8. The method according to claim 1, wherein the microbial cell is selected from the group consisting of a fungal cell and a bacterial cell.

9. The method according to claim 8, wherein the microbial cell is a Penicillium sp. cell.

10. The method according to claim 9, wherein the Penicillium sp. cell is a single strain of Penicillium having the accession number of CCM 8364.

11. The method according to claim 1, wherein the culture medium comprises a basic nitrogen source and a complex nitrogen source.

12. The method according to claim 11, wherein the complex nitrogen source is selected from hydrolyzed casein, corn steep or mixtures thereof.

13. The method according to claim 11, wherein the basic nitrogen source comprises an amino acid supplement.

14. The method according to claim 13, wherein the amino acid supplement is one or more of glycine, methionine and asparagine.

15. The method according to claim 1, wherein the level of oxygen saturation is controlled in the range 5 to 40%.

16. The method according to claim 1, wherein the level of oxygen saturation is controlled throughout the entire production phase.

17. The method according to claim 16, wherein the level of oxygen saturation in the culture medium is controlled during a time period selected from the time period from 0 to 24 hours, the time period from 0 to 48 hours, from 24 to 48 hours, and from 48 hours of inoculation.

18. The method according to claim 16, wherein the level of oxygen saturation is controlled until the pH rises due to carbon source depletion at the end of the production phase.

19. The method according to claim 1, wherein the culture medium is maintained at a pH greater than pH 4.

20. The method according to claim 19, wherein the pH is maintained by regulating the level of oxygen saturation in the culture medium during a production stage for the acid metabolite.

21. The method according to claim 20, wherein the pH is between about pH 4.5 and about pH 5.5 during the production stage.

22. A method of preparing mycophenolate mofetil comprising converting mycophenolic acid to mycophenolate mofetil, wherein the mycophenolic acid is prepared by regulating the oxygen saturation of a fermentation broth comprising a mycophenolic acid producing microbial cell.

23. A strain of Penicillium sp. deposited as accession number CCM 8364 at Czech Collection of Microorganisms (CCM) at Masaryk University, Bmo, Czech Republic.

24. The strain according to claim 23 in the form of individual cells, cell aggregates, hyphas, filaments and its aggregates in or on any surface or environment.

25. The strain of Pencillium sp. according to claim 23, wherein the strain is capable to produce a final concentration of MPA (g/l of culture medium) that is in excess of 1 g/l.

26. The strain of Pencillium sp. according to claim 25, wherein the concentration is in excess of 5 g/l.

27. The strain of Pencillium sp. according to claim 26, wherein the concentration is in excess of 8 g/l.

28. A method for producing an acid metabolite comprising fermenting the strain of claim 23.

29. The method according to claim 28, wherein the acid metabolite is selected from the group consisting of uronic acid, paspalic acid, methylcarbinolamine of lysergic acid, and mycophenolic acid.

30. The method according to claim 29, wherein the acid metabolite is mycophenolic acid (MPA).
